(19) United States
(12) Patent Application Publication
(10) Pub. No.: US 2026/0138937 A1
(43) Pub. Date: May 21, 2026

(54) CONTINUOUS PRODUCTION METHOD AND CONTINUOUS PRODUCTION DEVICE OF ISOBUTYLBENZENE

(71) Applicant: Qingdao University of Science and Technology, Qingdao City (CN)

(72) Inventors: Peizhe Cui, Qingdao City (CN); Xin Li, Qingdao City (CN); Guoxuan Li, Qingdao City (CN); Limei Zhong, Qingdao City (CN); Zhaoyou Zhu, Qingdao City (CN); Yinglong Wang, Qingdao City (CN); Kaiguang Wang, Qingdao City (CN); Chengshun Yang, Qingdao City (CN); Jinbin Fan, Qingdao City (CN); Wenhui Xu, Qingdao City (CN); Zuxing Kou, Qingdao City (CN); Zhonghui Zheng, Qingdao City (CN)

(21) Appl. No.: 19/029,669

(22) Filed: Jan. 17, 2025

(30) Foreign Application Priority Data
Nov. 18, 2024 (CN) 202411648449.9

(51) Int. Cl. C07C 2/72; B01J 8/00

(52) U.S. Cl. CPC C07C 2/72; B01J 8/0055; C07C 2523/04; C07C 2527/232

(57) ABSTRACT

Provided are a continuous production method and a continuous production device of isobutylbenzene. The continuous production method includes: continuously introducing a raw material A and a raw material B into a reactor, and conducting side chain alkylation reaction to obtain an isobutylbenzene-containing reaction liquid, where the raw material A includes toluene and a catalyst, and the raw material B includes propylene; continuously discharging the isobutylbenzene-containing reaction liquid, and subjecting the isobutylbenzene-containing reaction liquid to solid-liquid separation to obtain a solid and a first feed liquid; subjecting the first feed liquid to first rectification to obtain a second feed liquid and a first gas component; subjecting the second feed liquid to second rectification to obtain a third feed liquid and a second gas component; and subjecting the third feed liquid to third rectification, and liquefying to obtain isobutylbenzene.

### CROSS-REFERENCE TO RELATED APPLICATION

This patent application claims the benefit and priority of Chinese Patent Application No. 2024116484499 filed with the China National Intellectual Property Administration on Nov. 18, 2024, the disclosure of which is incorporated by reference herein in its entirety as part of the present application.

### TECHNICAL FIELD

The present disclosure belongs to the technical field of organic synthesis, and in particular to a continuous production method and a continuous production device of isobutylbenzene.

### BACKGROUND

Ibuprofen is a non-steroidal anti-inflammatory drug with analgesic effects in peripheral and central regions. It has been proved that ibuprofen is an extremely effective antipyretic and analgesic drug for adults and children, with small side effects. Isobutylbenzene, as an important substance in the production of ibuprofen, has a general structural formula of C₆H₅—CH₂—CH(CH₃)₂, is a colorless liquid, insoluble in water, soluble in organic solvents such as ethanol and ether, and has an aromatic smell and a boiling point of about 170-172° C.

At present, the isobutylbenzene is produced by a batch kettle reaction. Toluene, liquid propylene and a catalyst are added into a batch reactor at one time and mixed, and then heated for side chain alkylation to obtain isobutylbenzene. Discharging is carried out after the reaction is completed, the whole process is obviously intermittent and periodic. This traditional technology is relatively low in production efficiency, low in output per unit time, difficult in process control, easy to cause fluctuation, difficult to keep the consistency of the quality of different batches of products, and poor in stability of product quality; thus, it is not suitable for large-scale production. Moreover, this technology is not ideal in equipment utilization rate and energy consumption, which will cause resource waste and cost increase. In addition, the recovery of catalyst and raw materials is relatively difficult, and the conversion rate of raw materials is low, which is usually only about 40%.

### SUMMARY

In view of this, an object of the present disclosure is to provide a continuous production method and a continuous production device of isobutylbenzene. The continuous production method of isobutylbenzene provided by the present disclosure has high raw material conversion rate, less impurity generation in the reaction process, and high product yield.

The present disclosure provides a continuous production method of isobutylbenzene, including the following steps:
continuously introducing a raw material A and a raw material B into a reactor, and conducting side chain alkylation reaction to obtain an isobutylbenzene-containing reaction liquid, wherein the raw material A includes toluene and a catalyst, and the raw material B includes propylene;
continuously discharging the isobutylbenzene-containing reaction liquid, and subjecting the isobutylbenzene-containing reaction liquid to solid-liquid separation to obtain a solid and a first feed liquid, wherein the solid includes a recovered catalyst, which is recycled for the side chain alkylation reaction;
subjecting the first feed liquid to first rectification to obtain a second feed liquid and a first gas component, wherein the first gas component is mainly recovered propylene, which is recycled for the side chain alkylation reaction;
subjecting the second feed liquid to second rectification to obtain a third feed liquid and a second gas component, wherein the second gas component is mainly recovered toluene, which is recycled for the side chain alkylation reaction; and
subjecting the third feed liquid to third rectification to obtain a third gas component, and liquefying the third gas component to obtain the isobutylbenzene.

In some embodiments, the catalyst is a mixture of an alkali metal and an alkali metal carbonate, and a mass ratio of the alkali metal to the alkali metal carbonate in the catalyst is in a range of 0.5-1.5:1-3.

In some embodiments, a mass ratio of the toluene to the catalyst is in a range of 95-99.5:0.5-5.

In some embodiments, a mass ratio of the propylene to the toluene is in a range of 4-9:1-5.

In some embodiments, the side chain alkylation reaction is carried out at a temperature of 165-255° C.

In some embodiments, the side chain alkylation reaction is carried out at a pressure of 1-5 MPa.

In some embodiments, a residence time of the raw material A and the raw material B in the reactor is in a range of 15-30 min.

The present disclosure provides a continuous production device of isobutylbenzene, including:
a reaction kettle;
a first storage tank and a second storage tank communicating with a feed port of the reaction kettle, respectively;
a hydrocyclone communicating with a discharge port of the reaction kettle;
a first rectification column communicating with an overflow outlet of the hydrocyclone, wherein an underflow outlet of the hydrocyclone communicates with the reaction kettle;
a second rectification column communicating with a column-bottom discharge port of the first rectification column, wherein a column-top discharge port of the first rectification column communicates with the first storage tank; and
a third rectification column communicating with a column-bottom discharge port of the second rectification column, wherein a column-top discharge port of the second rectification column communicates with the second storage tank.

In some embodiments, the continuous production device of isobutylbenzene further includes a conveying pump connected between the discharge port of the reaction kettle and the hydrocyclone.

In some embodiments, the continuous production device of isobutylbenzene further includes a first compressor connected between the column-top discharge port of the first rectification column and the first storage tank; and
a second compressor is connected between the column-top discharge port of the second rectification column and the second storage tank.

Compared with the prior art, the present disclosure has the following beneficial effects:

The continuous production method includes the following steps: continuously introducing a raw material A and a raw material B into a reactor, and conducting side chain alkylation reaction to obtain an isobutylbenzene-containing reaction liquid, wherein the raw material A includes toluene and a catalyst, and the raw material B includes propylene; continuously discharging the isobutylbenzene-containing reaction liquid, and subjecting the isobutylbenzene-containing reaction liquid to solid-liquid separation to obtain a solid and a first feed liquid, wherein the solid includes a recovered catalyst, which is recycled for the side chain alkylation reaction; subjecting the first feed liquid to first rectification to obtain a second feed liquid and a first gas component, wherein the first gas component is mainly recovered propylene, which is recycled for the side chain alkylation reaction; subjecting the second feed liquid to second rectification to obtain a third feed liquid and a second gas component, wherein the second gas component is mainly recovered toluene, which is recycled for the side chain alkylation reaction; and subjecting the third feed liquid to third rectification to obtain a third gas component, and liquefying the third gas component to obtain the isobutylbenzene.

The continuous production method of isobutylbenzene of the present disclosure makes it possible to operate the reaction uninterruptedly through continuous feeding and discharging operations, and reduce side reactions such as self-polymerization of propylene and the generation of impurities. According to the continuous production method of the present disclosure, a rectification system could recover unreacted propylene and toluene after the reaction, and recycle the unreacted raw materials, thus improving the raw material conversion rate and product yield, significantly improving the utilization rate of resources, and reducing the waste of raw materials.

By adopting the continuous synthesis method of the present disclosure, the time for reaction can be precisely controlled, which is beneficial to control the reaction time, the feeding time and the discharging time, reduce the generation of impurities, and further improve the purity and yield of isobutylbenzene. By reasonably controlling the residence time, temperature, pressure and catalyst dosage of a reactant, a complete reaction of reactant is ensured, and a discharge of unreacted raw materials is reduced.

According to the present disclosure, the continuity of the production process is achieved, which could effectively improve production efficiency, shorten production cycle, and supply market demand rapidly and massively. Furthermore, the continuous and stable reaction environment is beneficial to precisely control the reaction condition, thus ensuring a high degree of consistency and stability of the product quality. The continuous production method provided by the present disclosure is of a great significance in resource utilization efficiency and energy saving, and could reduce production cost and impact on environment. The present disclosure brings brand-new changes to the production of isobutylbenzene, and promotes the development of related industries in a more efficient, high-quality and sustainable direction.

### BRIEF DESCRIPTION OF THE DRAWINGS

To describe the technical solutions of the embodiments of the present disclosure or in the prior art more clearly, the accompanying drawings used in the embodiments will be briefly described. Apparently, the accompanying drawings in the following description show merely some embodiments of the present disclosure, and those skilled in the art may derive other drawings from these accompanying drawings without creative efforts.

FIG. 1 is a schematic diagram of the device for continuously producing isobutylbenzene in an embodiment, in which S01 represents a propylene storage tank, S02 represents a toluene storage tank, R01 represents an isobutylbenzene synthesis kettle, P01 represents a conveying pump, F01 represents a hydrocyclone, T01 represents a rectification column 1, T02 represents a rectification column 2, T03 represents a rectification column 3, C01 represents a compressor 1, and C02 represents a compressor 2.

FIG. 2A is a diagram showing influence results of different reaction temperatures on a product yield.

FIG. 2B is a diagram showing influence results of different reaction pressures on a product yield.

FIG. 2C is a diagram showing influence results of different catalyst dosages on a product yield.

FIG. 2D is a diagram showing influence results of different residence time on a product yield.

### DETAILED DESCRIPTION OF THE EMBODIMENTS

The present disclosure provides a continuous production method of isobutylbenzene, including the following steps:
continuously introducing a raw material A and a raw material B into a reactor, and conducting side chain alkylation reaction to obtain an isobutylbenzene-containing reaction liquid, wherein the raw material A includes toluene and a catalyst, and the raw material B includes propylene;
continuously discharging the isobutylbenzene-containing reaction liquid, and subjecting the isobutylbenzene-containing reaction liquid to solid-liquid separation to obtain a solid and a first feed liquid, wherein the solid includes a recovered catalyst, which is recycled for the side chain alkylation reaction;
subjecting the first feed liquid to first rectification to obtain a second feed liquid and a first gas component, wherein the first gas component is mainly recovered propylene, which is recycled for the side chain alkylation reaction;
subjecting the second feed liquid to second rectification to obtain a third feed liquid and a second gas component, wherein the second gas component is mainly recovered toluene, which is recycled for the side chain alkylation reaction; and
subjecting the third feed liquid to third rectification to obtain a third gas component, and liquefying the third gas component to obtain the isobutylbenzene.

In the present disclosure, unless otherwise specified, all materials used and equipment are commercially available commodities in the art.

According to the present disclosure, the raw material A and the raw material B are continuously introduced into the reactor for side chain alkylation reaction to obtain an isobutylbenzene-containing reaction liquid. The raw material A includes toluene and a catalyst, and the raw material B includes propylene.

In the present disclosure, in some embodiments, the catalyst is a mixture of an alkali metal and an alkali metal carbonate. In some embodiments, the catalyst is a mixture of potassium and sodium carbonate, or a mixture of sodium and potassium carbonate. In some embodiments, a mass ratio of the alkali metal to the alkali metal carbonate in the catalyst is in a range of 0.5-1.5:1-3, and preferably 0.8:1.2.

In the present disclosure, in some embodiments, a mass ratio of the toluene to the catalyst is in a range of 95-99.5:0.5-5, and preferably 96-99:1-4. In some embodiments, a mass ratio of the toluene to the catalyst is 99:1, 98:2, or 97:3. In some embodiments, the content of the catalyst in the raw material A is in a range of 1 wt % to 3 wt %. In some embodiments, the content of the catalyst in the raw material A is 1 wt %, 2 wt %, or 3 wt %.

In the present disclosure, in some embodiments, a mass ratio of the propylene to the toluene is in a range of 4-9:1-5. In some embodiments, a mass ratio of the propylene to the toluene is 1:1.

In the present disclosure, in some embodiments, the side chain alkylation reaction is carried out at a temperature of 165-255° C., and preferably 180-250° C. In some embodiments, the side chain alkylation reaction is carried out at 180° C., 190° C., 195° C., 200° C., 210° C., 225° C., 240° C., or 250° C.

In the present disclosure, in some embodiments, the side chain alkylation reaction is carried out at a pressure of 1-5 MPa. In some embodiments, the side chain alkylation reaction is carried out at 2 MPa, 3 MPa, 4 MPa, or 5 MPa.

In the present disclosure, in some embodiments, the residence time of the raw material A and the raw material B is in a range of 15-30 min. In some embodiments, the residence time of the raw material A and the raw material B is 20 min, or 25 min.

After the isobutylbenzene-containing reaction liquid is obtained, the isobutylbenzene-containing reaction liquid is continuously discharged, and subjected to solid-liquid separation to obtain a solid and a first feed liquid. The solid includes a recovered catalyst, which is recycled for the side chain alkylation reaction.

In the present disclosure, the purpose of the solid-liquid separation is to achieve the separation of the catalyst and the feed liquid, and then the catalyst can be recovered and recycled for the side chain alkylation reaction.

The first feed liquid is subjected to first rectification to obtain a second feed liquid and a first gas component. The first gas component is mainly recovered propylene, which is recycled for the side chain alkylation reaction.

In the present disclosure, in some embodiments, a rectification column used for the first rectification is at a pressure of 3.5 MPa, with a column-top temperature of 90-100° C., and a column-bottom temperature of 105-115° C. The temperature and pressure of the first rectification can vaporize propylene (a boiling point of propylene is about 95° C. at 3.5 MPa), while other components remain in the feed liquid, thus achieving a separation of propylene. In some embodiments, after the first rectification, the method further includes liquefying the obtained first gas component.

The second feed liquid is subjected to second rectification to obtain a third feed liquid and a second gas component. The second gas component is mainly recovered toluene, which is recycled for the side chain alkylation reaction.

In the present disclosure, in some embodiments, a rectification column used for the second rectification is at a column-top temperature of 110-115° C., and a column-bottom temperature of 120-130° C. The temperature of the second rectification can vaporize toluene (a boiling point of propylene is about 110.6° C. at a normal pressure), while other components remain in the feed liquid, thus achieving a separation of toluene. In some embodiments, after the second rectification, the method further includes liquefying the obtained second gas component.

The third feed liquid is subjected to third rectification, and an obtained third gas component is liquefied to obtain isobutylbenzene.

In the present disclosure, a rectification column used for the third rectification is at a column-top temperature of 165-170° C., and a column-bottom temperature of 175-185° C. The temperature of the third rectification can vaporize isobutylbenzene (a boiling point of isobutylbenzene is about 169° C. at a normal pressure), while other components with high boiling point remain in the feed liquid, thus achieving a separation of isobutylbenzene.

The present disclosure further provides a continuous production device of isobutylbenzene, including a reaction kettle;
a first storage tank and a second storage tank communicating with a feed port of the reaction kettle, respectively;
a hydrocyclone communicating with a discharge port of the reaction kettle;
a first rectification column communicating with an overflow outlet of the hydrocyclone, where an underflow outlet of the hydrocyclone communicates with the reaction kettle;
a second rectification column communicating with a column-bottom discharge port of the first rectification column, where a column-top discharge port of the first rectification column communicates with the first storage tank; and
a third rectification column communicating with a column-bottom discharge port of the second rectification column, where a column-top discharge port of the second rectification column communicates with the second storage tank.

In the present disclosure, the reaction kettle is used for the synthesis of isobutylbenzene, the first storage tank is used for storing propylene, and the second storage tank is used for storing toluene.

In the present disclosure, in some embodiments, the continuous production device further includes a conveying pump connected between the discharge port of the reaction kettle and the hydrocyclone for conveying materials. The hydrocyclone is used to separate a feed liquid from the catalyst, and the catalyst is recycled for catalyzing the synthesis of isobutylbenzene.

In the present disclosure, in some embodiments, the continuous production device further includes a first compressor connected between the column-top discharge port of the first rectification column and the first storage tank, which is used to liquefy propylene; and a second compressor connected between the column-top discharge port of the second rectification column and the second storage tank, which is used to liquefy toluene.

To further describe the present disclosure, the continuous production method and device of isobutylbenzene provided by the present disclosure will be described in detail below with reference to the accompanying drawings and examples, but the descriptions cannot be understood as limiting the scope of the present disclosure.

In examples 1 to 3 of the present disclosure, the raw material A was composed of 98 wt % toluene, 0.8 wt % potassium, and 1.2 wt % sodium carbonate. A mixture of the potassium and sodium carbonate were used as a catalyst. Propylene was used as the raw material B. The capacity of the synthesis kettle is 5000 L.

In the examples, the used device is as shown in FIG. 1, in which S01 represents a propylene storage tank, S02 represents a toluene storage tank, R01 represents an isobutylbenzene synthesis kettle, P01 represents a conveying pump, F01 represents a hydrocyclone, T01 represents a rectification column 1, T02 represents a rectification column 2, T03 represents a rectification column 3, C01 represents a compressor 1, and C02 represents a compressor 2.

### Example 1

The device was mounted and debugged according to FIG. 1. Toluene and the catalyst were mixed to obtain a mixed liquid (the content of the catalyst in the mixed liquid was 2 wt %) as a raw material A, and propylene was used as a raw material B. The raw material A and the raw material B were conveyed to a synthesis kettle (R01). A feeding proportion was that a mole ratio of toluene to propylene was 1:1. When the material was fed to half the volume of the reaction kettle, a temperature in the synthesis kettle was heated to 195° C. by using a heat transfer oil, and the reaction begins in the kettle, where a pressure in the synthesis kettle was 3 MPa. After reacting for 30 min, the feeding and discharging of the synthesis kettle were started at the same time, and the residence time of the material in the synthesis kettle was 30 min. The material at a discharge port was conveyed to a hydrocyclone through a conveying pump (P01), a separation of the catalyst and the feed liquid was achieved after the material entered the hydrocyclone, and then the catalyst was recovered for a second time and utilized. The feed liquid was conveyed to a rectification column 1 (T01), which has an operating pressure of 3.5 MPa, a column-top temperature of 90-100° C., and a column-bottom temperature of 105-115° C. Propylene with the lowest boiling point in the feed liquid was extracted from the top of the column, liquefied by a compressor (C01), and then recovered to a propylene storage tank (S01) for recycling. The rest of the feed liquid was conveyed to a rectification column 2 (T02), which has a column-top temperature of 110-115° C., and a column-bottom temperature of 120-130° C. Toluene in the feed liquid was extracted from the top of the column, liquefied by a compressor (C02), and then recovered to a toluene storage tank (S02) for recycling. The rest of the feed liquid was conveyed to a rectification column 3 (T03) to separate isobutylbenzene from impurities with a high boiling point. The column-top temperature of the rectification column 3 was 165-170° C. and the column-bottom temperature was 175-185° C. Finally, the isobutylbenzene product was obtained at the top of T03. The reaction lasted for 8 hours.

In this example, the conversion rate of toluene is 72%, and the yield of isobutylbenzene is 39.5%.

### Example 2

Example 2 was carried out according to example 1, expect that the temperature in the synthesis kettle was heated to 210° C., and the pressure in the synthesis kettle was 5 MPa.

In this example, the conversion rate of toluene is 60.5%, and the yield of isobutylbenzene is 35.2%.

### Example 3

Example 3 was carried out according to example 1, expect that the temperature in the synthesis kettle was heated to 180° C., and the pressure in the synthesis kettle was 1 MPa.

In this example, the conversion rate of toluene is 48.6%, and the yield of isobutylbenzene is 33.6%.

The combination of appropriate temperature and medium pressure in example 1 provides the highest conversion rate and yield, indicating that for this reaction, it is not the higher the temperature and pressure, the better. By comparing the three examples, it can be seen that the combination of medium temperature and medium pressure is the best, while the effects of high temperature and pressure (Example 2) and low temperature and pressure (Example 3) are not good.

### Example 4

Orthogonal experiments were conducted to investigate the effects of reaction temperature, reaction pressure, catalyst dosage (a mass ratio of potassium to sodium carbonate was kept at 2:3) and material residence time on the synthesis of isobutylbenzene. Except for the variables in the table, other conditions were the same as those in Example 1, and setting conditions and results were shown in Tables 1 and 2.

TABLE 1
Influence of reaction temperature and reaction
pressure on synthesis of isobutylbenzene

| Test | | | Conversion | |
|---|---|---|---|---|
| number | Temperature/° C. | Pressure/MPa | rate/% | Yield/% |
| 1 | 165 | 1 | 40.5 | 31.1 |
| 2 | | 2 | 50.3 | 34.6 |
| 3 | | 3 | 66.2 | 36.1 |
| 4 | | 4 | 60.3 | 35.3 |
| 5 | | 5 | 51.3 | 33.4 |
| 6 | 180 | 1 | 48.6 | 33.6 |
| 7 | | 2 | 55.3 | 36.6 |
| 8 | | 3 | 60.6 | 37.2 |
| 9 | | 4 | 66.7 | 35.5 |
| 10 | | 5 | 55.2 | 33.3 |
| 11 | 195 | 1 | 60.2 | 35 |
| 12 | | 2 | 66.3 | 37.5 |
| 13 | | 3 | 72 | 40.5 |
| 14 | | 4 | 69.8 | 39 |
| 15 | | 5 | 66.4 | 37.9 |
| 16 | 210 | 1 | 50.6 | 33.3 |
| 17 | | 2 | 55.6 | 36.6 |
| 18 | | 3 | 62.3 | 37.6 |
| 19 | | 4 | 63.3 | 37.2 |
| 20 | | 5 | 60.5 | 35.2 |
| 21 | 225 | 1 | 55.6 | 32.2 |
| 22 | | 2 | 59.3 | 35.3 |
| 23 | | 3 | 62.3 | 37.1 |
| 24 | | 4 | 66.8 | 36.1 |
| 25 | | 5 | 62.3 | 33.5 |
| 26 | 240 | 1 | 52.6 | 31.3 |
| 27 | | 2 | 58.2 | 34.3 |
| 28 | | 3 | 64.3 | 35.7 |
| 29 | | 4 | 68.1 | 34.2 |
| 30 | | 5 | 63.4 | 32.5 |
| 31 | 255 | 1 | 54.1 | 30.4 |
| 32 | | 2 | 61.3 | 33.7 |
| 33 | | 3 | 66.8 | 34.5 |
| 34 | | 4 | 69.5 | 32.8 |
| 35 | | 5 | 64.9 | 31.2 |

TABLE 2
Influence of catalyst dosage and residence
time on synthesis of isobutylbenzene

| Content of | | | |
|---|---|---|---|
| catalyst in raw | Residence time | | |
| material A (wt %) | (min) | Conversion rate/% | Yield/% |
| 1 | 25 | 54 | 31.2 |
| | 30 | 60.5 | 34.1 |
| | 100 | 61.2 | 35.3 |
| 2 | 25 | 67.5 | 38.2 |
| | 30 | 71.9 | 40.1 |
| | 100 | 72.5 | 41.0 |
| 3 | 25 | 58.1 | 36.1 |
| | 30 | 69.3 | 38.2 |
| | 100 | 69.8 | 39.7 |

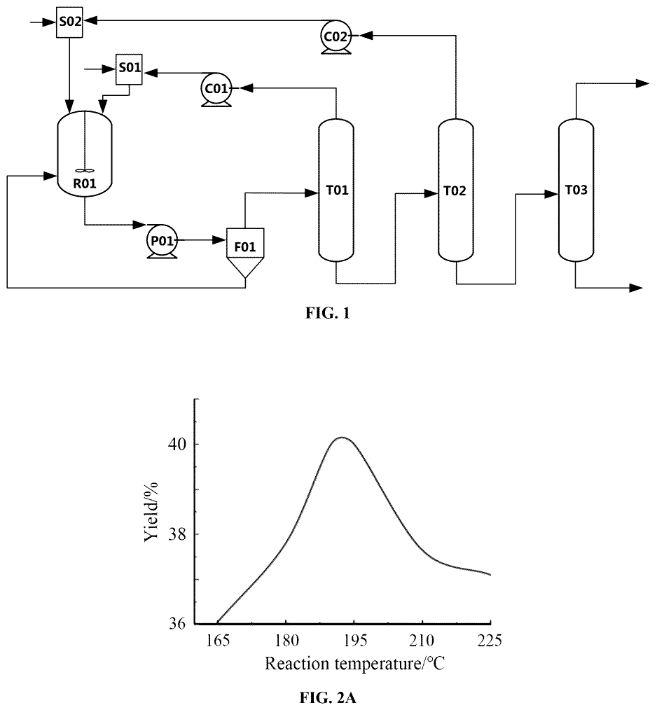

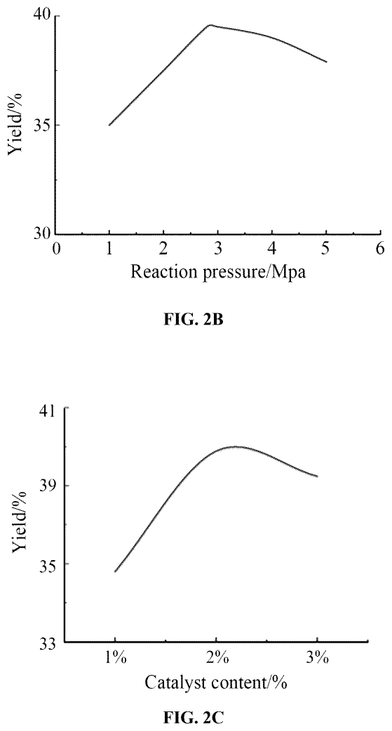

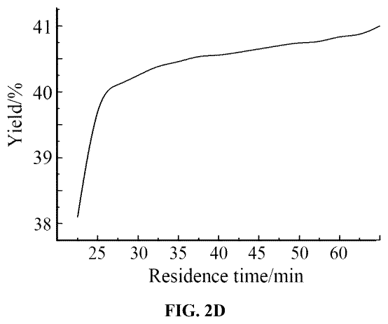

Influence results of different catalyst dosages, different reaction temperatures, different reaction pressures and different residence time on a product yield are shown in FIG. 2A to FIG. 2D. From FIG. 2A to FIG. 2D, it can be seen that with the increase of the catalyst dosage, the product yield gradually increases. When the catalyst content in the raw material A reaches 2 wt %, the product yield is the highest, and then with the increase of the catalyst content, the product yield begins to decline. With the increase of the reaction temperature, the product yield gradually increases. When the reaction temperature reaches 195° C., the product yield is the highest, and then with the increase of the reaction temperature, the product yield begins to decline. With the increase of the reaction pressure, the product yield gradually increases. When the reaction pressure reaches 3 MPa, the product yield is the highest, and then with the increase of the reaction pressure, the product yield begins to decline. With the increase of the residence time, the product yield gradually increases. When the residence time exceeds 30 min, the yield grows slowly. Thus, it is determined that the optimal residence time is 30 min.

Compared with the existing batch isobutylbenzene production technology, the continuous kettle production adopted by the present disclosure has obvious advantages. The existing batch production technology has the problems of discontinuous operation, low efficiency, tedious process, and difficulty in precise control. The continuous kettle production of the present disclosure can achieve uninterrupted flow, improve the production efficiency and output, and control various process parameters more accurately at the same time, thus ensuring the stability and consistency of the product quality. Through the continuous production mode, the pause and conversion time of intermediate links are effectively reduced, the overall production efficiency is greatly improved, and many disadvantages of the existing batch production technology are successfully solved. By adopting the continuous production, the reaction pressure is lower than the traditional batch reaction pressure, and the unreacted raw materials can be recycled.

Although the above examples have described the present disclosure in detail, these examples are only a part rather than all of the examples of the present disclosure. Those skilled in the part can also obtain other embodiments according to the embodiments of the present disclosure without creative labor, and these embodiments all belong to the scope of the present disclosure.

1-13. (canceled)

14. A continuous production method of isobutylbenzene, comprising the following steps:
continuously introducing a raw material A and a raw material B into a reactor, and conducting side chain alkylation reaction to obtain an isobutylbenzene-containing reaction liquid, wherein the raw material A comprises toluene and a catalyst, and the raw material B comprises propylene; the catalyst in the raw material A has a content of 2 wt % to 3 wt %; a molar ratio of the propylene to the toluene is 1:1; the side chain alkylation reaction is carried out at a temperature of 195° C. and a pressure of 3 MPa; a residence time of the raw material A and the raw material B in the reactor is 30 min; and the catalyst is a mixture of an alkali metal and an alkali metal carbonate, and a mass ratio of the alkali metal to the alkali metal carbonate in the catalyst is in a range of 0.5-1.5:1-3;
continuously discharging the isobutylbenzene-containing reaction liquid, and subjecting the isobutylbenzene-containing reaction liquid to solid-liquid separation to obtain a solid and a first feed liquid, wherein the solid comprises a recovered catalyst, which is recycled for the side chain alkylation reaction;
subjecting the first feed liquid to first rectification to obtain a second feed liquid and a first gas component, wherein the first gas component is mainly recovered propylene, which is recycled for the side chain alkylation reaction;
subjecting the second feed liquid to second rectification to obtain a third feed liquid and a second gas component, wherein the second gas component is mainly recovered toluene, which is recycled for the side chain alkylation reaction; and
subjecting the third feed liquid to third rectification to obtain a third gas component, and liquefying the third gas component to obtain the isobutylbenzene;
wherein a continuous production device for the continuous production method of the isobutylbenzene comprises:
a reaction kettle;
a first storage tank and a second storage tank communicating with a feed port of the reaction kettle, respectively;
a hydrocyclone communicating with a discharge port of the reaction kettle;
a first rectification column communicating with an overflow outlet of the hydrocyclone, wherein an underflow outlet of the hydrocyclone communicates with the reaction kettle;
a second rectification column communicating with a column-bottom discharge port of the first rectification column, wherein a column-top discharge port of the first rectification column communicates with the first storage tank; and
a third rectification column communicating with a column-bottom discharge port of the second rectification column, wherein a column-top discharge port of the second rectification column communicates with the second storage tank.

15. The continuous production method of claim 14, wherein the continuous production device further comprises a conveying pump connected between the discharge port of the reaction kettle and the hydrocyclone.

16. The continuous production method of claim 14, wherein the continuous production device further comprises:
a first compressor connected between the column-top discharge port of the first rectification column and the first storage tank; and
a second compressor connected between the column-top discharge port of the second rectification column and the second storage tank.

17. The continuous production method of claim 15, wherein the continuous production device further comprises:
a first compressor connected between the column-top discharge port of the first rectification column and the first storage tank; and
a second compressor connected between the column-top discharge port of the second rectification column and the second storage tank.
